(19) United States
(12) Patent Application Publication
(10) Pub. No.: US 2008/0287447 A1
(43) Pub. Date: Nov. 20, 2008

(54) Methods for preparing eszopiclone

(76) Inventors: Nina Finkelstein, Herzliya (IL); Marioara Mendelovici, Rehovot (IL); Revital Ramaty, Ramat-Hasharon (IL); Alex Mainfeld, Kfar-Saba (IL)

Correspondence Address: KENYON & KENYON LLP, ONE BROADWAY, NEW YORK, NY, 10004, US

(21) Appl. No.: 12/012,422

(22) Filed: Jan. 31, 2008

Related U.S. Application Data
(60) Provisional application No. 60/898,405, filed on Jan. 31, 2007.
(60) Provisional application No. 60/929,682, filed on Jul. 9, 2007.

(51) Int. Cl. A61K 31/4985; C07D 471/04; A61P 25/20

(52) U.S. Cl. 514/249; 544/350

(57) ABSTRACT

Methods for the preparation of crystalline eszopiclone free base in water, in the absence of organic solvents, are provided. The methods are more environmentally acceptable than prior art methods, and produce eszopiclone free base essentially free of residual organic solvent.

### CROSS REFERENCE TO RELATED APPLICATIONS

The present application claims the benefit of the following U.S. Provisional Patent Application Nos. 60/898,405, filed Jan. 31, 2007; 60/929,682, filed Jul. 9, 2007; and U.S. patent application Ser. No. 11/738,115, filed Apr. 20, 2007. The contents of these applications are incorporated herein by reference.

### FIELD OF THE INVENTION

The present invention relates to methods for preparing eszopiclone.

### BACKGROUND

Zopiclone, a non-benzodiazepine which can be used to induce a sedative, hypnotic or tranquilizing effect, useful for treating insomnia, is a racemate having a chemical name of 4-methyl-1-piperazinecarboxylic acid 6-(5-chloro-2-pyridinyl)-6,7-dihydro-7-oxo-5H-pyrrolo[3,4-b]pyrazin-5-yl ester, (±)-6-(5-chloro-2-pyridinyl)-6,7-dihydro-7-oxo-5H-pyrrolo[3,4-b]pyrazin-5-yl-4-methylpiperazine-1-carboxylate, or 6-(5-chloropyrid-2-yl)-5-(4-methylpiperazin-1-yl)carbonyloxy-7-oxo-6,7-dihydro-5H-pyrrolo[3,4-b]pyrazine, represented with formula I below.

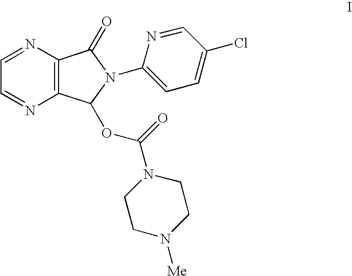

Eszopiclone is the S-enantiomer of zopiclone, and is more active and less toxic than the racemic zopiclone according to U.S. Pat. No. 6,444,673 B1. This drug, formerly known as Estorra®, is marketed in the United States by Sepracor™ under the name Lunesta®. Eszopiclone has the chemical name (+)-6-(5-chloro-2-pyridinyl)-7(S)-(4-methylpiperazin-1-yl-carbonyloxy)-6,7-dihydro-5H-pyrrolo[3,4-b]pyrazine-5-one, CAS Registry Number 138729-47-2, and is represented by formula II below.

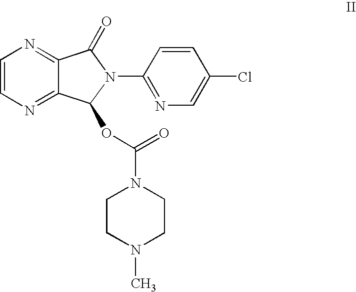

Eszopiclone in free base form and salt form are disclosed in U.S. Pat. Nos. 6,444,673 and 6,864,257.

Eszopiclone can be prepared by optical resolution of racemic zopiclone. U.S. Pat. No. 6,444,673 discloses a process for preparing eszopiclone free base by preparing the D-(+)-O,O′-dibenzoyl-tartarate salt followed by two crystallizations; neutralizing the eszopiclone salt with aqueous sodium hydroxide in the presence of dichloromethane to obtain a two phase system; separating the organic phase; evaporating the organic phase to dryness; and crystallizing the product from acetonitrile.

Blaschke, G. et al., Chirality (1993) 5:419-421 disclose preparation of eszopiclone free of its enantiomer using 0.5 equivalent D-(+)-malic acid. By this procedure the diastereomeric salt is crystallized from a mixture of methanol-acetone, the salt is then neutralized with KHCO₃ and the free base is extracted into CH₂Cl₂/ethyl acetate and precipitated by concentration of the solution.

The preparation of eszopiclone by optical resolution of racemic zopiclone using malic acid was improved in U.S. Pat. No. 6,339,086 using one equivalent D-(+)-malic acid. This patent discloses a method for eszopiclone free base preparation by neutralizing eszopiclone D-(+)-malate salt with aqueous K₂CO₃ in a mixture of water and ethyl acetate, heating the mixture to about 65° C.; isolating the organic phase; concentrating; and isolating.

US 2005/0043311 discloses optical resolution with D-(+)-dibenzoyl-tartaric acid to obtain the eszopiclone salt, neutralization of the salt in the presence of methylene chloride and aqueous NaOH, separating the organic phase and evaporating it to obtain eszopiclone. The eszopiclone is then crystallized from acetonitrile.

The use of a variety of organic solvents to recrystallize eszopiclone has been disclosed in US 2007/0270590. The solvents examined led to crystalline eszopiclone having residual organic solvent, even after vacuum drying. The present application claims the benefit of a U.S. provisional application filed before the publication of US 2007/0270590

There is a need in the art for methods for the preparation of eszopiclone free base that will reduce the volume of solvents used in the process, use more environmentally friendly solvents, and conserve energy and improve yields by operating at lower temperatures. There is also a need for methods of producing eszopiclone with reduced levels of residual organic solvents.

### SUMMARY OF THE INVENTION

In one embodiment, the present invention provides a method for obtaining Eszopiclone comprising combining at least one salt of Eszopiclone with water and at least one base.

In one embodiment, the present invention provides a method for obtaining Eszopiclone comprising reacting a salt of eszopiclone with a base in one phase reaction mixture comprising water to obtain eszopiclone.

In another embodiment, the present invention provides a process for preparing eszopiclone comprising reacting a salt of eszopiclone with a base in water in the absence of an organic solvent to obtain eszopiclone.

In another embodiment, the present invention provides a process for improving the color of eszopiclone by an activated carbon treatment comprising dissolving a salt of eszopiclone in water; and adding activated carbon.

In another embodiment, the present invention provides eszopiclone having less than about 5000 ppm residual organic solvent.

### DETAILED DESCRIPTION OF THE INVENTION

As used herein, “eszopiclone” refers to eszopiclone free base.

As used herein, the term “ambient temperature” refers to a temperature of about 20° C. to about 25° C.

As used herein, the term “mild base” refers to a base having a pKa of about 13 or less, preferably about 12 to about 6, more preferably about 12 to about 9. Preferred bases are selected from the group consisting of ammonia, mono-, di- or tri-alkylamines (preferably wherein each alkyl group contains from 1 to 6 carbon atoms and more preferably from 1 to 3 carbon atoms, and more preferably is methyl or ethyl), alkali metal carbonates, alkaline-earth metal carbonates, alkali metal bicarbonates, and alkaline earth metal bicarbonates, and combinations thereof. More preferred are the alkali metal carbonates and alkali metal bicarbonates, most preferably sodium and potassium carbonate and sodium and potassium bicarbonate. It is preferred that the cationic species in the base will produce a water-soluble salt when paired with the anionic species present in the eszopiclone salt.

Processes in the art for the preparation of eszopiclone from its D-(+)-malate salt or its D-(+)-dibenzoyl-tartarate salt include extraction of eszopiclone from an aqueous phase, in a two phase system under basic conditions. The processes described in the art, such as in U.S. Pat. No. 6,339,086, require large quantities of solvents in order to extract the material from the aqueous phase. In order to reduce the solvent volume, the extraction is done at higher temperatures. Consequently, the yields of these processes are low due to the degradation of the compound under the basic conditions and under heating. In addition, the optical purity of the product is affected since an isomerization process occurs under basic conditions and under heating. Also, the concentration of the material from ethyl acetate leads to a high residual solvent.

The present invention provides an improved method for obtaining eszopiclone that comprises neutralization of a salt of eszopiclone, particularly eszopiclone D-(+)-malate, in water with a base, preferably with a mild base, at ambient temperature, avoiding the heating in the presence of the base as in the prior art procedures. The obtained eszopiclone is isolated directly by filtration from water, a step which minimizes the exposure of the material to heat and/or base. Optionally, a charcoal treatment may be performed before the base addition in order to improve the color. The process of the present invention allows for production at industrial scale with greater yields in comparison to the processes in the prior art.

The process for obtaining eszopiclone comprises combining at least one salt of eszopiclone with water; and adding at least one base. It is also possible to first add the base to the water and then combine eszopiclone with the water. The solvent consists essentially of water resulting one phase reaction mixture. An organic solvent is not used in the process, especially, a water immiscible organic solvent.

A preferred concentration of salt of eszopiclone in water is preferably about 0.01 to about 0.2 by g/ml. A preferred molar ratio of the base to the salt is preferably about 0.9 to about 3 by mol/mol.

The salt can be acidic salt (organic or inorganic), preferably the salt of optically active acid. The salt can be water soluble salt. Most preferably, the salt is a water soluble salt of optically active acid, such as D-(+)-malate salt. Additional examples of organic optically active acidic salts are: D-(+)-O,O-ditoluoyl-tartarate, D-(+)-tartarate, D-(+)-mandelate, and D-(+)-O,O′-dibenzoyl tartarate. When the salt is not a water soluble salt, a slurry is obtained. When the salt is a water soluble salt, a solution is obtained.

Preferably, the temperature during reaction of the base with the salt is of about 5° C. to about 60° C., more preferably, of about ambient temperature to about 50° C., more preferably, about ambient temperature to about 40° C., and, most preferably, about ambient temperature.

Preferably, prior to the base addition, activated carbon is added to the solution. Preferably, the solution containing the activated carbon is stirred. Preferably, the stirring is for about ½ hour to about 5 hours, more preferably, for about ½ hour to about 3 hours, and most preferably, for about 1 hour. Preferably, after the stirring and prior to the base addition, the activated carbon is removed. Preferably, the removal is by filtration.

Preferably, the base is a mild base, organic and inorganic. More preferable, the mild base is inorganic base. The base can be selected from the group consisting of ammonia, alkaline earth hydroxide, alkali metal hydroxide, alkali metal carbonates, alkaline earth carbonates, alkali metal bicarbonates alkaline earth bicarbonates and amines. Preferably, the alkali metal is selected from the group consisting of potassium and sodium. Preferably, the carbonate is selected from the group consisting of potassium carbonate and sodium carbonate. Preferably, the bicarbonate is sodium bicarbonate or potassium bicarbonate. The at least one inorganic base may be added as a solid or an aqueous solution. Preferably, the at least one inorganic base is added as an aqueous solution. Preferably, the aqueous solution of the at least one inorganic base is added gradually. Preferably, the aqueous solution of the at least one inorganic base is added during a period of about 1 hour to about 3 hours, more preferably, about 2 hours to about 3 hours.

An ideal pH after addition of the base is about 7 to about 12, preferably about 8.

After the base addition a suspension is obtained. Preferably, the suspension is stirred for a period of time after addition of the base. Preferably, the stirring is for about 1 hour to about 24 hours, and more preferably, for about 2 hours to about 4 hours.

Preferably, the obtained eszopiclone is further isolated. Preferably, the isolation is by filtration. The isolated eszopiclone may be further washed and dried. Preferably, the washing is with water. Preferably, the drying is at a temperature of about 30° C. to about 80° C., more preferably, about 40° C. to about 70° C., most preferably, about 50° C., under vacuum (less than 100 mmHg). Preferably, the drying is for about 6 hours to about 16 hours.

The present invention further provides a process for improving the color of eszopiclone by an activated carbon treatment. The process comprises dissolving a salt of eszopiclone, in water; and adding activated carbon. Preferably, the salt of eszopiclone is a water soluble salt and more preferably a water soluble salt of an optically active acid such as malate salt. By improving color, it is meant to remove colored impurities so that the eszopiclone obtained is more white.

The parameters of this process are as described above.

The processes of the present invention can produce crystalline eszopiclone. A crystalline form is Form A, disclosed in US 2007/0270590, incorporated herein by reference. Eszopiclone Form A is crystalline eszopiclone characterized by the following main XRD peaks: 5.1, 10.1, 11.3, 12.6, 16.1, 18.1, 19.1, 20.2, 21.4, 25.7, 27.7.+−0.0.2 degrees 2 theta.

The present invention also provides eszopiclone having residual organic solvent less than the amount recommended for pharmaceutical products, as set forth for example in ICH guidelines and U.S. pharmacopoeia; the recommended amount is less than 5000 ppm for ethyl acetate and i-butyl acetate; less than 800 ppm for toluene and less than 5000 ppm for isopropyl alcohol. Preferably, the amount is less than about 5000 ppm residual organic solvent, preferably, more preferably less than about 2000 ppm residual organic solvent, most preferably, less than about 700 ppm.

The optical purity of the eszopiclone is preferably more than about 99.9%.

Pharmaceutical compositions can be prepared by combining eszopiclone having a low residual solvent with at least one pharmaceutically acceptable carrier. The pharmaceutical compositions can be made into dosage forms, such as tablets and capsules, and administered to a patient to induce sleep. Eszopiclone having a low residual solvent can also be used in the manufacture of a medicament for inducing sleep.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### Experimental Methods and Instruments:

HPLC method for chemical purity determination:
HPLC

| Column & packing | Inertsil ODS 3 V 250*4.6 mm 5 μ C.N 5020- | | |
|---|---|---|---|
| | 01802 | | |
| Buffer preparation: | 0.01M Sodium dihydrogen Phosphate | | |
| | adjusted to pH = 7.0 with 1 N of NaOH | | |
| Eluent A: | 66% Buffer:34% Acetonitrile | | |
| Eluent B: | Acetonitrile | | |
| Gradient of Eluent: | Time (min) | Eluent A (%) | Eluent B (%) |
| | 0 min | 100 | 0 |
| | 13 min | 100 | 0 |
| | 23 min | 40 | 60 |
| | 33 min | 40 | 60 |

| | |
|---|---|
| Stop time: | 33 min |
| Equilibration time: | 7 min |
| Flow: | 1.0 ml/min |
| Detector: | 306 nm. |
| Injection volume: | 20 l. |
| Diluent | 50% Acetonitrile:50% Buffer |
| Column temperature | 250° C. |
| Autosampler temp. | 100° C. |
| Column & packing | DAICEL Chiralcel OD-H 250*4.6 mm 5 μm |
| | CN: 14325 |
| Eluent: | 0.1% DEA in Ethanol |
| Stop time: | 25 min |
| Flow: | 0.7 ml/min |
| Detector: | 306 nm. |
| Injection volume: | 20 μl. |
| Diluent | Ethanol |
| Column temperature | 25° C. |
| Autosampler | 10° C. |
| temperature | |

Chromatographic Conditions for measuring the residual solvents amount:

| Column: | DB-624, 30 m × 0.53 mm ID, 3 μm film thickness | |
|---|---|---|
| | (J & W 125-1334) or equivalent. | |
| Carrier gas: | Helium about 3.7 psi, constant pressure | |
| | (about 5 mL/min. at 40° C). | |
| Injection mode: | Headspace, split | |
| Split Ratio: | 1:5 by using COMBI PAL (CTC Analytics) headspace | |
| | sampler (gas-syringe system) | |
| | 1:20 by using HP-7694/Agilent G1888 headspace | |
| | sampler system (pressure/loop system) | |
| Detector: | Flame Ionization Detector. | |
| Make up gas: | Helium, about 25 mL/min. | |
| Temperature: | Injector: | 180° C. |
| | Detector: | 260° C. |
| | Oven program: | Initial temperature: 40° C. |
| | Initial time: | 3.0 min. |

| | | |
|---|---|---|
| Rate | Final temp. | Final time |
| 15° C./min. | 140° C. | 5.0 min |

### Headspace Injection System

Use one of the headspace sampler systems

### 2.1. Combi Pal (CTC Analytics) Headspace Sampler (Gas-Syringe System)

| | |
|---|---|
| Syringe: | 2.5 mL |
| Sample volume: | 1 mL |
| Incub. Temperature: | 80° C. |
| Incub. Time: | 35 min. |
| Agi Speed: | 500 rpm |
| Agi on time: | 5 s |
| Agi off time: | 5 s |
| Syringe temp.: | 100° C. |
| Fill speed: | 300 μL/s |
| Pull up del.: | 1 s |
| Inject speed: | 800 μL/s |
| Pre Inj. del.: | 0 s |
| Post inj. del.: | 1.5 s |
| Syr. Flushing: | 2.5 min. |
| G.C. run time: | 24 min.* |

### 2.2. HP-7694/Agilent G1888 Headspace Sampler (Pressure/Loop System)

| | | |
|---|---|---|
| Vial pressure: | 12.5 psi | |
| Temperature: | Oven: | 80° C. |
| | Loop: | 100° C. |
| | Transfer line: | 110° C. |
| Times: | G.C. cycle: | 24 min* |
| | Sample eq.: | 35 min. |
| | Pressurize: | 0.20 min. |
| | Loop fill: | 0.10 min. |
| | Loop eq.: | 0.05 min. |
| | Injection: | 0.50 min. |
| Shaking: | 1 (low) | |
| Loop volume: | 1 mL | |

*Note:
G.C. Cycle time is a function of room temperature, which may be modified accordingly.

### Sample Preparation

The sample is dissolved in Dimethylsulfoxide about 100 mg in 1 mL.

### EXAMPLES

### Example 1

### Neutralization Process for Obtaining Eszopiclone Having an Improved Color from D-(+)-Eszopiclone Malate

To a solution of D-(+)-eszopiclone malate (1.0 g, 1.87 mmol, optical purity 98.01%; purity profile 99.91%) in 20 ml of water activated carbon (0.1 g) was added. The mixture was stirred at 25° C. for an hour. The activated carbon was removed by filtration, and the mother liquor was basified by addition of 40% aqueous potassium carbonate (0.6 ml, 2.3 mmol) with stirring. The suspension was stirred for 2 hours at 25° C. The solid was filtered, washed with water, dried under vacuum at 50° C. overnight and gave 0.65 g (90%) of eszopiclone (optical purity 98.12%, Chemical purity profile 99.94%).

### Example 2

### Neutralization Process for Obtaining Eszopiclone from D-(+)-Eszopiclone Malate

A solution of D-(+)-eszopiclone malate (5.0 g, 9.35 mmol, optical purity 98.46%; purity profile 99.92%) in 100 ml of water was basified by addition of 40% aqueous potassium carbonate (2.9 ml, 11.6 mmol) with stirring. The suspension was stirred for 4 hours at 30° C. The solid was filtered, washed with water, dried under vacuum at 50° C. overnight and gave 3.3 g (91%) of eszopiclone (optical purity 98.12%, Chemical purity profile 99.94%).

### Example 3

### Neutralization Process for Obtaining Eszopiclone from D-(+)-Eszopiclone Malate

A solution of D-(+)-eszopiclone malate (5.0 g, 9.35 mmol, optical purity 98.46%; purity profile 99.92%) in 100 ml of water was basified by addition of 40% aqueous potassium carbonate (2.9 ml, 11.6 mmol) with stirring. The suspension was stirred for 2 hours at 40° C. The solid was filtered, washed with water, dried under vacuum at 50° C. overnight and gave 3.2 g (88%) of eszopiclone (optical purity 98.20%, Chemical purity profile 99.96%).

### Example 4

### Neutralization Process for Obtaining Eszopiclone from D-(+)-Eszopiclone Malate

A solution of D-(+)-eszopiclone malate (5.0 g, 9.35 mmol, optical purity 98.46%; purity profile 99.92%) in 100 ml of water was basified by addition of 40% aqueous potassium carbonate (2.9 ml, 11.6 mmol) with stirring. The suspension was stirred for 2 hours at 25° C. The solid was filtered, washed with water, dried under vacuum at 50° C. overnight and gave 3.3 g (91%) of eszopiclone (optical purity 98.12%, Chemical purity profile 99.94%).

### Example 5

### Neutralization Process for Obtaining Eszopiclone from D-(+)-Eszopiclone Malate

A solution of D-(+)-eszopiclone malate (5.0 g, 9.35 mmol, optical purity 98.46%; purity profile 99.92%) in 100 ml of water was basified by addition of 25% aqueous sodium carbonate (4.0 ml, 11.6 mmol) with stirring. The suspension was stirred for 2 hours at 25° C. The solid was filtered, washed with water, dried under vacuum at 50° C. overnight and gave 3.5 g (97%) of eszopiclone (optical purity 98.15%, Chemical purity profile 99.94%).

### Example 6

### Neutralization Process for Obtaining Eszopiclone from D-(+)-Eszopiclone Malate

A solution of D-(+)-eszopiclone malate (5.0 g, 9.35 mmol, optical purity 98.46%; purity profile 99.92%) in 100 ml of water was basified by addition of 40% aqueous potassium carbonate (2.6 ml, 10.3 mmol) with stirring. The suspension was stirred for 2 hours at 25° C. The solid was filtered, washed with water, dried under vacuum at 50° C. overnight and gave 3.3 g (91%) of eszopiclone (optical purity 98.17%, Chemical purity profile 99.95%).

### Example 7

### Neutralization Process for Obtaining Eszopiclone from D-(+)-Eszopiclone Malate

A solution of D-(+)-eszopiclone malate (5.0 g, 9.35 mmol, optical purity 98.46%; purity profile 99.92%) in 100 ml of water was basified by addition of 40% aqueous potassium carbonate (3.5 ml, 14.0 mmol) with stirring. The suspension was stirred for 2 hours at 25° C. The solid was filtered, washed with water, dried under vacuum at 50° C. overnight and gave 3.4 g (94%) of eszopiclone (optical purity 98.17%, Chemical purity profile 99.91%).

### Example 8

### Neutralization Process for Obtaining Eszopiclone from D-(+)-Eszopiclone Malate

A solution of D-(+)-eszopiclone malate (5.0 g, 9.35 mmol, optical purity 98.46%; purity profile 99.92%) in 75 ml of water was basified by addition of 40% aqueous potassium carbonate (2.9 ml, 11.6 mmol) with stirring. The suspension was stirred for 2 hours at 25° C. The solid was filtered, washed with water, dried under vacuum at 50° C. overnight and gave 3.4 g (94%) of eszopiclone (optical purity 98.12%, Chemical purity profile 99.95%).

### Example 9

### Neutralization Process for Obtaining Eszopiclone from D-(+)-Eszopiclone Malate

A solution of D-(+)-eszopiclone malate (5.0 g, 9.35 mmol, optical purity 98.46%; purity profile 99.92%) in 50 ml of water was basified by addition of 40% aqueous potassium carbonate (2.9 ml, 11.6 mmol) with stirring. The suspension was stirred for 2 hours at 25° C. The solid was filtered, washed with water, dried under vacuum at 50° C. overnight and gave 3.4 g (94%) of eszopiclone (optical purity 98.12%, Chemical purity profile 99.94%).

### Example 10

### Neutralization Process for Obtaining Eszopiclone from D-(+)-Eszopiclone Malate

A solution of D-(+)-eszopiclone malate (8.0 g, 18.6 mmol, optical purity 98.46%; purity profile 99.92%) in 160 ml of water was basified by addition of solid sodium carbonate (1.97 g, 18.6 mmol) with stirring. The suspension was stirred for 2 hours at 25° C. The solid was filtered, washed with water, dried under vacuum at 50° C. overnight and gave 5.4 g (93%) of eszopiclone (optical purity 98.33; Chemical purity profile 99.93%)

### Example 11

### Neutralization Process for Obtaining Eszopiclone from D-(+)-Eszopiclone Malate

A solution of D-(+)-eszopiclone malate (24.6 g, 46 mmol) in 250 ml of water was basified by addition of 40% aqueous potassium carbonate (14.3 ml, 57 mmol) with stirring. The suspension was stirred for 20 minutes at 25° C. The solid was filtered, washed with water, dried under vacuum at 50° C. overnight and gave 17 g (95%) of eszopiclone (Chemical purity profile 99.98%), having crystal form A.

### Example 12

### Neutralization Process for Obtaining Eszopiclone from D-(+)-Eszopiclone Malate by Slow Addition of the Base

To a solution of D-(+)-eszopiclone malate (15 g, 29 mmol) in 300 ml of water was added sodium carbonate (3.76 g, 35 mmol) with stirring in portions during 1 hour. During the addition the temperature rose from the room temperature to about 35° C. The suspension was stirred for 2 h at 25° C. The solid was filtered, washed with water, and dried under vacuum at 40° C. overnight, providing 9.59 g (yield 90.5%) of eszopiclone.

### Example 13

### Repetition of Example 2 from U.S. Pat. No. 6,339,086

To a mixture of eszopiclone malate (2.0 g, 3.74 mmol, purity profile 99.96% by HPLC and optical purity 96% by HPLC) in water (4 ml) and ethyl acetate (20 ml), 40% aqueous potassium carbonate (1.6 g, 4.64 mmol) was added slowly with stirring at 30° C. Then the mixture was heated at 60° C., and the organic phase was isolated and washed with 20 ml of water. The mixture was concentrated to ⅔ volume of the organic solvent. The resulted slurry was cooled to 5° C. and stirred at the same temperature for additional 2 hours. The solid was filtered, washed with cold ethyl acetate, dried at 50° C. under vacuum overnight (the first drying) yielding eszopiclone containing residual ethyl acetate at a level of 7534 ppm (GC) with an optical purity of 96.35% by HPLC. The eszopiclone was further dried at 75° C. under vacuum for 18 hours (the second drying) yielding eszopiclone containing residual ethyl acetate at a level of 7360 ppm (GC).

### Example 14

### Using Other Organic Solvents to Conduct a Process Similar to the Process in Example 2 of U.S. Pat. No. 6,339,086

To a mixture of eszopiclone malate (2.0 g, 3.74 mmol, purity profile 99.96% by HPLC and optical purity 96%) in water (4 ml) and one of the organic solvents shown in the table below, 40% aqueous potassium carbonate (1.6 g, 4.64 mmol) was added slowly with stirring at 30° C. Then the mixture was heated, and the organic phase was isolated and washed with 20 ml of water. The mixture was concentrated. The resulted slurry was cooled to 5° C. and stirred at the same temperature for additional 2 hours. The solid was filtered, washed with cold organic solvent (as used before), dried at 50° C. under vacuum overnight (the first drying) to obtain eszopiclone containing residual solvent. The eszopiclone was further dried at 75° C. under vacuum for 18 hours (the second drying), again resulting in eszopiclone containing residual solvent.

The results of Examples 13 and 14 (involving preparation of eszopiclone from eszopiclone malate by neutralization, extraction with the organic solvent and precipitation) are shown in the table below.

| | Volume of | | | | | | | |
|---|---|---|---|---|---|---|---|---|
| | organic | | | | | | | |
| | solvent | Heating | | | | | Residual | Residual |
| | in ml per | temperature | | | | | solvent | solvent |
| | gram of | (during the | | | Chemical | Optical | (ppm) | (ppm) |
| Organic | starting | extraction) | Yield | Recovery* | purity | purity | After 1^{st} | After 2^{nd} |
| Solvent | eszopiclone | (° C.) | (%) | (%) | (%) | (%) | drying | drying |
| Ethyl | 20 | 60 | 80 | | | 96.35 | 7534 | 7360 |
| acetate | | | | | | | | |
| Butyl | 54 | 80 | 73 | Not | Not | 96.4 | 6733 | 6468 |
| acetate | | | | checked | analyzed | | | |
| Toluene | 25 | 80 | 75 | Not | 99.95 | 96.8 | 8025 | 7478 |
| | | | | checked | | | | |
| iso- | 64 | 80 | 60 | 83 | 99.94** | 99.15 | 7799 | 7578 |
| butyl | | | | | | | | |
| acetate | | | | | | | | |

*The “Recovery” relates to the total amount of eszopiclone (in its solid form and in the mother liquor)
**Material balance of eszopiclone: Yield of the solid and the material in mother liquids in these experiments was less than 83%, while the purity profile of the material dissolved in mother liquids was about 95%, indicating decomposition of eszopiclone. In the water phase, eszopiclone was absent.

### Example 15

### Crystallization of Eszopiclone from Different Organic Solvents

The starting material in Example 15 was prepared according to one of examples 1-11. The results of this experiment are shown in the table below. (The experiment is purification by crystallization; after neutralization of eszopiclone salt (usually eszopiclone malate in water in the presence of a base), eszopiclone is filtrated and crystallized from an organic solvent.)

Residual solvent in eszopiclone prepared by crystallization

| | | Chemical | | Optical | |
|---|---|---|---|---|---|
| Solvent, | | purity of | Optical purity | purity of | |
| volume in relation to | | eszopiclone | of starting | eszopiclone | Residual |
| starting eszopiclone | Yield | product | eszopiclone | product | solvent |
| (ml/gr) | (%) | (%) | (%) | (%) | (ppm) |
| toluene (9) | 90 | 99.97 | 98.85 | 99.96 | 1349 |
| isobutyl acetate (27) | 80 | 99.95 | 98.85 | 99.97 | 568 |
| isopropanol/water, | 85 | 99.97 | 95.50 | 99.91 | 676 |
| volume ratio 5:1, | | | | | |
| (14) | | | | | |

### Example 16

### Preparation of Eszopiclone from Eszopiclone Malate by Neutralization in Water, Filtration and Crystallization from Organic Solvent

To a mixture of eszopiclone malate (2.0 g, 3.74 mmol, purity profile 99.96% by HPLC and optical purity 96%) in water (4 ml), 40% aqueous potassium carbonate (1.6 g, 4.64 mmol) was added slowly with stirring at 30° C. to obtain a precipitate. The solid was filtered, washed with water, dried at 50° C. under vacuum overnight (the first drying) to obtain eszopiclone. The eszopiclone was further dried at 75° C. under vacuum for 18 hours (the second drying), resulting in eszopiclone containing residual solvent. The eszopiclone was further crystallized from an organic solvent resulting in eszopiclone containing residual solvent according to the table below. The product was further dries at 50° C. resulting in crystallized eszopiclone containing residual solvent according to the table below. The results of this experiment are shown in the table below

Residual solvent in Eszopiclone prepared by crystallization

| | | Chemical | | Optical | |
|---|---|---|---|---|---|
| Solvent, | | purity of | Optical purity | purity of | |
| volume in relation to | | Eszopiclone | of starting | Eszopiclone | Residual |
| starting Eszopiclone | Yield | product | Eszopiclone | product | solvent |
| (ml/gr) | (%) | (%) | (%) | (%) | (ppm) |
| toluene (9) | 90 | 99.97 | 98.85 | 99.96 | 1349 |
| isobutyl acetate (27) | 80 | 99.95 | 98.85 | 99.97 | 568 |
| isopropanol/water, | 85 | 99.97 | 95.5 | 99.91 | 676 |
| volume ratio 5:1, | | | | | |
| (14) | | | | | |

What is claimed is:

1. Eszopiclone having less than about 5000 ppm residual organic solvent.

2. The eszopiclone of claim 1 having less than about 2000 ppm residual organic solvent.

3. The eszopiclone of claim 2 having less than about 700 ppm residual organic solvent.

4. The eszopiclone of claim 1, wherein the organic solvent is i-butyl acetate, or isopropyl alcohol.

5. A process for preparing eszopiclone comprising reacting a salt of eszopiclone with a base in one phase reaction mixture comprising water to obtain eszopiclone.

6. The process of claim 5, wherein the process is carried out in the absence of an organic solvent.

7. The process of claim 5, wherein the process is carried out in the absence of a water immiscible organic solvent.

8. The process of claim 4, wherein the salt of eszopiclone is acidic salt.

9. The process of claim 4, wherein the eszopiclone is optically active acidic salt.

10. The process of claim 4, wherein the salt of eszopiclone is water soluble salt.

11. The process of claim 4, wherein the salt of eszopiclone is selected from the group consisting of: D-(+)-O,O-ditoluoyl-tartarate, D-(+)-tartarate, D-(+)-mandelate, D-(+)-O,O′-dibenzoyl tartarate and D-(+)-malate salt.

12. The process of claim 4, wherein the salt of eszopiclone is D-(+)-malate salt.

13. The process of claim 4, wherein the temperature during reaction of the base with the salt is of about 5° C. to about 50° C.

14. The process of claim 4, wherein the base is a mild base.

15. The process of claim 4, wherein the base is organic or inorganic base.

16. The process of claim 4, wherein the base is selected from the group consisting of ammonia, alkali metal hydroxides, alkaline earth hydroxides, alkali metal carbonates, alkaline earth carbonates, alkali metal bicarbonates, alkaline earth bicarbonates and amines.

17. The process of claim 16, wherein the alkali metal is selected from the group consisting of potassium and sodium.

18. The process of claim 4, wherein the base is selected from the group consisting of potassium carbonate and sodium carbonate.

19. The process of claim 4, wherein the base is sodium bicarbonate or potassium bicarbonate.

20. The process of claim 4, wherein pH after addition of the base is about 7 to about 12.

21. The process of claim 20, wherein the pH is about 8.

22. The process of claim 4, further comprising isolating the eszopiclone.

23. The process of claim 4, wherein prior to the base addition, activated carbon is added to the solution.

24. The process of claim 4, wherein the eszopiclone has less than 5000 ppm residual organic solvent.

25. The process of claim 24, wherein the eszopiclone has less than about 2000 ppm residual organic solvent.

26. The process of claim 25, wherein the eszopiclone has less than about 700 ppm residual organic solvent.

27. The process of claim 4, wherein the eszopiclone has an optical purity that is more than about 99.9%.

28. A process for improving the color of eszopiclone by an activated carbon treatment comprising dissolving a salt of D-(+)-eszopiclone in water; and adding activated carbon.

29. The process of claim 28, wherein the salt of eszopiclone is D-(+)-O,O-ditoluoyl-tartarate, D-(+)-tartarate D-(+)-mandelate, D-(+)-malate and D-(+)-O,O′-dibenzoyl tartarate.

30. A pharmaceutical composition comprising the eszopiclone of claim 1 and at least one pharmaceutically acceptable carrier.

31. A method of inducing sleep in a patient comprising administering the pharmaceutical composition of claim 30 to the patient.
